(19) United States
(12) Patent Application Publication
(10) Pub. No.: US 2022/0288115 A1
(43) Pub. Date: Sep. 15, 2022

(54) ORAL DISODIUM PYROPHOSPHATE FOR USE IN REDUCING CALCIFICATION

(71) Applicant: STICHTING HET NEDERLANDS KANKER INSTITUT-ANTONI VAN LEEUWENHOEK ZIEKENHUIS, Amsterdam (NL)

(72) Inventors: Suvi Johanna VÄÄRÄMÄKI, Amsterdam (NL); Pasi Ilari NEVALAINEN, Amsterdam (NL); András VÁRADI, Amsterdam (NL)

(21) Appl. No.: 17/626,741

(22) PCT Filed: Jul. 9, 2020
(86) PCT No.: PCT/NL2020/050451

§ 371 Date: Jan. 12, 2022

(30) Foreign Application Priority Data
Jul. 12, 2019 (NL) 2023491

(51) Int. Cl. A61K 33/42; A61K 9/00; A61P 3/14

(52) U.S. Cl. CPC A61K 33/42; A61K 9/0053; A61P 3/14

(57) ABSTRACT

The current invention relates to use of oral disodium pyrophosphate for preventing and/or reducing tissue calcification, particularly soft tissue calcification, and/or diseases or disorders characterized by low plasma PPi levels, as, e.g., occurs in chronic kidney disease (CKD), end-stage renal disease (ESRD), generalized arterial calcification of infancy (GACI), Pseudoxanthoma elasticum (PXE), Arterial Calcification Due to Deficiency of CD73 (ACDC), Ehlers-Danlos syndrome, arteriosclerosis obliterans, venous calcifications, crystal deposition disorders, calcification resulting from neurological disorders, calcinosis universalis, calcinosis circumscripta, scleroderma, dermatomyositis, systemic lupus erythematosus, hyperparathyroidism, neoplasms, milk-alkali syndrome, hypervitaminosis D, tumoral calcinosis, hypophosphatemic rickets, ossification of the posterior longitudinal ligament of the spine, myocardial ischemia, joint calcification, heterotropic ossification of traumatized muscle, angioid streaks, diabetes mellitus type II, cardiovascular disorder, calciphylaxis, calciphylaxis secondary to chronic kidney disease, calcific uremic arteriolopathy or atherosclerosis.

### FIELD OF THE INVENTION

The present invention is in the field of calcification, particularly tissue calcification, more particularly soft tissue calcification, and treatment thereof. This invention also relates to diseases or disorders the treatment of that would benefit from increasing inorganic pyrophosphate plasma levels, including such as diseases or disorders characterized by low inorganic pyrophosphate plasma levels, but also diseases or disorders characterized by normal inorganic pyrophosphate plasma levels.

### BACKGROUND OF THE INVENTION

Physiological mineralization is essential for the normal development of vertebrates. It is restricted to specific sites of the body. In mammals, biominerals predominantly consist of calcium and phosphate, together forming hydroxyapatite. In plasma and several other body fluids calcium and phosphate are present at concentrations that by far exceed their solubility constant. Vertebrates have evolved mechanisms to stabilize this supersaturated solution and to allow the regulated precipitation of calcium and phosphate only at specific bodily compartments.

Calcification (deposits of calcium phosphate) may occur in many different soft tissues in a variety of local and systemic (throughout the body) conditions. Calcium phosphate crystals have a remarkable tendency to aggregate into snowball-like clumps and are invariably associated with particular collagens. Collagens are fibrous, insoluble proteins found in the connective tissues, including skin, ligaments, and cartilage. Collagen represents about 30 percent of the total body protein.

Pyrophosphate (PPi) is a central factor in prevention of precipitation of calcium and phosphate in soft peripheral tissues. The liver is the most important source of circulatory PPi, via a pathway depending on ABCC6-mediated ATP release. Outside the hepatocytes, but still within the liver vasculature, released ATP is rapidly converted into AMP and PPi by the ectoenzyme ectonucleotide pyrophosphatase phosphodiesterase 1 (ENPP1). Pyrophosphate is a potent inhibitor of hydroxyapatite formation, and, under normal conditions, functions to inhibit soft tissue calcification, e.g., vascular calcification.

Inactivating mutations in the genes encoding the enzymes involved in PPi homeostasis result in rare hereditary calcification disorders. For example, absence of functional ABCC6 results in pseudoxanthoma elasticum (PXE), a late onset ectopic calcification disorder, with lesions found in the skin, eyes and cardiovascular system. Biallelic inactivating mutations in ENPP1 cause arterial calcification and generalized calcification of infancy (GACI), a condition that can become life-threatening shortly after birth due to massive calcification of the large and medium-sized arteries. GACI patients have virtually no PPi in their blood, which explains the severity of the disease.

As reduced PPi concentrations in the circulation underlie the ectopic calcification disorders PXE and GACI, an obvious treatment for these disorders as well as other disorders characterized by reduced PPi concentrations in the circulation would be PPi supplementation. Due to the necessity to, in some instances, treat patients life-long and the short half-life of PPi, oral administration would be preferred for such a treatment. However, it has long been thought, and is therefore a reigning dogma, that PPi is ineffective when given orally (H. Fleisch, et al., Calc. Tiss. Res. 2, Suppl. (1968) 10; Francis, et al. Science (1969), 165(3899), 1264-1266; Orriss, I R., et al. Curr Opin Pharmacol. (2016) 28, 57-68). In contrast to this reigning dogma, International application WO2018052290 shows that, in animal model experiments, PPi provided to drinking water is effective when given orally.

In light of this, further or improved products, compositions, methods and uses for preventing and/or treating diseases or disorders characterized by calcification, particularly tissue calcification, particularly soft tissue calcification, or diseases or disorders the treatment of which would benefit from increased inorganic plasma levels, including, but not limited to diseases or disorders characterized by low plasma PPi levels would be highly desirable, but are not yet readily available. In particular there is a clear need in the art for reliable, efficient and reproducible products, compositions, methods and uses that allow effective amounts of PPi to be provided to subjects in need thereof, and that have, for example, limited side-effects and/or that are easily administered to such subjects, without causing substantive discomfort. Accordingly, the technical problem underlying the present invention can been seen in the provision of such products, compositions, methods and uses for complying with any of the aforementioned needs. The technical problem is solved by the embodiments characterized in the claims and herein below.

### SUMMARY OF THE INVENTION

In a first aspect, the present disclosure relates to disodium pyrophosphate for use as a medicament, and wherein said disodium pyrophosphate is administered in oral form. The disodium pyrophosphate as an oral medicament can suitably be administered to a subject having a disease or disorder, or that is at the risk of developing such disease or disorder, that can be prevented or be treated by increasing plasma levels of inorganic pyrophosphate. Such diseases or disorder may be characterized by normal plasma levels of inorganic pyrophosphate or my low plasma levels of inorganic pyrophosphate. Also provided is disodium pyrophosphate for use in preventing and/or treating diseases or disorders characterized by calcification, particularly tissue calcification, particularly soft tissue calcification, or diseases or disorders characterized by low plasma inorganic pyrophosphate (PPi) levels, wherein said disodium pyrophosphate is administered in oral form.

The soft tissue calcification may be vascular calcification such as arterial calcification or intimal calcification. The tissue calcification may be in a subject having ENPP1 deficiency, chronic kidney disease (CKD), end-stage renal disease (ESRD), generalized arterial calcification of infancy (GACI), Pseudoxanthoma elasticum (PXE), Arterial Calcification Due to Deficiency of CD73 (ACDC), Ehlers-Danlos syndrome, arteriosclerosis obliterans, venous calcifications, crystal deposition disorders, calcification resulting from neurological disorders, calcinosis universalis, calcinosis circumscripta, scleroderma, dermatomyositis, systemic lupus erythematosus, hyperparathyroidism, neoplasms, milk-alkali syndrome, hypervitaminosis D, tumoral calcinosis, hypophosphatemic rickets, ossification of the posterior longitudinal ligament of the spine, myocardial ischemia, joint calcification, heterotropic ossification of traumatized muscle, angioid streaks, diabetes mellitus type II, cardiovascular disorder, calciphylaxis, calciphylaxis secondary to chronic kidney disease, calcific uremic arteriolopathy, or atherosclerosis.

In other words, there is provided for disodium pyrophosphate for use in preventing and/or treating diseases or disorders wherein such diseases or disorders is characterized by calcification, particularly tissue calcification, particularly soft tissue calcification, or low plasma inorganic pyrophosphate (PPi) levels, wherein said disodium pyrophosphate is administered in oral form, preferably wherein the soft tissue calcification is vascular calcification such as arterial calcification or intimal calcification, preferably wherein said disease or disorder is selected from the group consisting of chronic kidney disease (CKD), end-stage renal disease (ESRD), generalized arterial calcification of infancy (GACI), Pseudoxanthoma elasticum (PXE), Arterial Calcification Due to Deficiency of CD73 (ACDC), Ehlers-Danlos syndrome, arteriosclerosis obliterans, venous calcifications, crystal deposition disorders, calcification resulting from neurological disorders, calcinosis universalis, calcinosis circumscripta, scleroderma, dermatomyositis, systemic lupus erythematosus, hyperparathyroidism, neoplasms, milk-alkali syndrome, hypervitaminosis D, tumoral calcinosis, hypophosphatemic rickets, ossification of the posterior longitudinal ligament of the spine, myocardial ischemia, joint calcification, heterotropic ossification of traumatized muscle, angioid streaks, diabetes mellitus type II, cardiovascular disorder, calciphylaxis, calciphylaxis secondary to chronic kidney disease, calcific uremic arteriolopathy, or atherosclerosis.

In a preferred embodiment the diseases or disorder characterized by tissue calcification is selected from the group consisting of PXE, GACI, calciphylaxis and calciphylaxis secondary to chronic kidney disease.

The disodium pyrophosphate may advantageously be administered to a human subject. The disodium pyrophosphate may be administered daily. The daily dose may be between 10-500 mg disodium pyrophosphate per kilogram bodyweight.

In a second aspect, the present disclosure provides a method for preventing and/or reducing calcification, particularly tissue calcification, particularly soft tissue, calcification, and/or diseases or disorders characterized by low plasma PPi levels comprising the step of: administering to a subject in need thereof a therapeutically effective amount of disodium pyrophosphate, wherein said disodium pyrophosphate is administered in oral form.

The soft tissue calcification may be vascular calcification such as arterial calcification or intimal calcification. The disodium pyrophosphate may be sufficient to achieve a transient increase in plasma PPi level in the subject. The transient increase in plasma PPi level may be characterized by a PPi level that is at least about 40% of the plasma PPi level in a healthy subject. The transient increase in plasma PPi level may be maintained for at least about 15 minutes. The subject may have a disease or disorder characterized by low plasma PPi levels, e.g., chronic kidney disease (CKD), end-stage renal disease (ESRD), generalized arterial calcification of infancy (GACI), Pseudoxanthoma elasticum (PXE), Arterial Calcification Due to Deficiency of CD73 (ACDC), Ehlers-Danlos syndrome, arteriosclerosis obliterans, venous calcifications, crystal deposition disorders, calcification resulting from neurological disorders, calcinosis universalis, calcinosis circumscripta, scleroderma, dermatomyositis, systemic lupus erythematosus, hyperparathyroidism, neoplasms, milk-alkali syndrome, hypervitaminosis D, tumoral calcinosis, hypophosphatemic rickets, ossification of the posterior longitudinal ligament of the spine, myocardial ischemia, joint calcification, heterotropic ossification of traumatized muscle, angioid streaks, diabetes mellitus type II, cardiovascular disorder, calciphylaxis, calciphylaxis secondary to chronic kidney disease, calcific uremic arteriolopathy or atherosclerosis. In a preferred embodiment the diseases or disorder characterized by tissue calcification is selected from the group consisting of PXE, GACI, calciphylaxis and calciphylaxis secondary to chronic kidney disease.

In an embodiment, the subject has GACI or PXE. A daily dose disodium pyrophosphate may be between 10-500 mg disodium pyrophosphate per kilogram bodyweight.

### DETAILED DESCRIPTION OF THE INVENTION

### Brief Description of the Drawings

Embodiments of the invention are further described hereinafter with reference to the accompanying drawings, in which:

FIG. 1: Plasma bioavailability of PPi after oral disodium PPi and after oral tetrasodium PPI. Results of different subjects (#) are shown, as well as AUC and AUC/mg PPi.

FIG. 2: Plasma PPi levels of the PXE-patient following oral PPi administration (disodium PPi). Dotted horizontal lines indicate the normal range of plasma PPi observed in healthy human volunteers. Legend indicates the dose (per kg of body weight) and time of PPi consumption.

### DEFINITIONS

The section headings used herein are for organizational purposes only and are not to be construed as limiting the subject matter described.

A portion of this disclosure contains material that is subject to copyright protection (such as, but not limited to, diagrams, device photographs, or any other aspects of this submission for which copyright protection is or may be available in any jurisdiction.). The copyright owner has no objection to the facsimile reproduction by anyone of the patent document or patent disclosure, as it appears in the Patent Office patent file or records, but otherwise reserves all copyright rights whatsoever.

Various terms relating to the methods, compositions, uses and other aspects of the present invention are used throughout the specification and claims. Such terms are to be given their ordinary meaning in the art to which the invention pertains, unless otherwise indicated. Other specifically defined terms are to be construed in a manner consistent with the definition provided herein. Although any methods and materials similar or equivalent to those described herein can be used in the practice for testing of the present invention, the preferred materials and methods are described herein.

For purposes of the present invention, the following terms are defined below.

As used herein, the term “subject” includes both mammals and non-mammals. Examples of mammals include, but are not limited to, humans, chimpanzees, apes, monkeys, cattle, horses, sheep, goats, swine, rabbits, dogs, cats, rats, mice, guinea pigs, and so on. Examples of non-mammals include, without limitation, birds, fish, and the like.

As used herein, the term “therapeutically effective amount” refers to a non-toxic amount of disodium pyrophosphate that is sufficient to result in improved treatment or healing of a disease or disorder, or a decrease in the rate of advancement of a disease or disorder.

The term “soft tissue” as used herein refers to the tissues that connect, support, or surround other structures and organs of the body, not being hard tissue such as bone. Soft tissue includes tendons, ligaments, fascia, skin, fibrous tissues, fat, and synovial membranes (which are connective tissue), and muscles, nerves and blood vessels (which are not connective tissue).

The term “about” as used herein is meant to denote variations of ±20% or ±10%, or ±5% or ±1% from the specified value, as such variations are still suitable to perform the methods taught herein.

The term “treating” as used herein refers to the administration of PPi in the form of disodium pyrophosphate to a subject who has a diseases or disorder that can be prevented or be treated by increasing plasma inorganic pyrophosphate levels, in particular to a subject who has a disease or disorder characterized by low PPi levels in the blood (plasma), or other progressive disorder characterized by the accumulation of deposits of calcium and other minerals, with the purpose of curing, healing, alleviating, relieving, altering, remedying, ameliorating, preventing, improving, or affecting the disease or disorder. The term “treating” refers to any indicia of success in the treatment or amelioration of an injury, pathology or condition, including any objective or subjective parameter such as abatement; remission; diminishing of symptoms; increasing tolerability of the injury, pathology or condition; slowing progression of the injury, pathology or condition; slowing the rate of degeneration or decline; or improving the subject's physical or mental well-being. Treatment may be therapeutic or prophylactic.

The term “preventing and/or reducing” as used herein refers to the prevention of calcification, the prevention of further calcification in (soft) tissues that already contain some degree of calcification as well as (partial) reversal of calcification already formed.

### DETAILED DESCRIPTION

It is contemplated that any method, use or composition described herein can be implemented with respect to any other method, use or composition described herein. Embodiments discussed in the context of methods, use and/or compositions of the invention may be employed with respect to any other method, use or composition described herein. Thus, an embodiment pertaining to one method, use or composition may be applied to other methods, uses and compositions of the invention as well.

As embodied and broadly described herein, the present invention is directed to the surprising finding that disodium pyrophosphate (e.g. Na₂H₂P₂O₇) when administered in oral form, has high bioavailability and efficiently increases plasma PPi in (human) subjects. It was found that, in comparison to other (salt) forms of pyrophosphate, oral disodium pyrophosphate at a substantial lower dose (expressed as PPi equivalent) achieves comparable or even higher plasma levels of PPi in subjects. As consequence thereof also sodium intake is reduced relative to other (slat) forms of pyrophosphate. In other words, higher concentrations of plasma PPi can be obtained when equimolar amounts of pyrophosphate are administered as disodium pyrophosphate in comparison to other forms of pyrophosphate.

Improvement of plasma PPi concentration can be achieved with a lower dose disodium pyrophosphate, reducing potential side-effects/adverse effects and/or discomfort to the subject (including lower rate of gastrointestinal tract related complaints: nausea, vomiting, diarrhea, and gastric dumping) or allowing to increase total dose without increase in such undesired effects, in comparison to other forms of pyrophosphate. Common mentioned side effects include GI complaints such as nausea, vomiting, diarrhea, gastric dumping.

### Methods of Treatment

The present invention relates to use of orally administered disodium pyrophosphate for treatment of calcification, for example tissue calcification, particularly in soft tissue calcification, e.g., vascular calcification. The reigning, over 50 year old, dogma has been that pyrophosphate has to be injected as it is ineffective orally because of hydrolytic destruction within the gut (Orriss et al. 2016, supra). In contrast to this reigning dogma, International application WO2018052290 shows that, in animal model experiments, PPi provided to drinking water is effective when given orally and can increase plasma PPi.

It has now surprisingly be found that oral disodium pyrophosphate has high bioavailability and that oral disodium pyrophosphate is highly effective in increasing plasma concentration of PPi, and in comparison to other forms of pyrophosphate. Oral disodium pyrophosphate is thus effective in increasing plasma PPi concentration and can attenuate calcification in subjects, including subjects with PXE. Hence, the present inventors have proven that orally administered disodium pyrophosphate can reach the blood circulation in subjects and counteract soft tissue calcification, making long-term treatment of soft tissue calcification disorders and diseases, some of which are hereditary and require life-long treatment, feasible. In other words, oral disodium pyrophosphate can be used to treat subjects that would benefit from an increased plasma inorganic pyrophosphate level. This may be subjects characterized by low plasma PPi levels, but may also be subjects that are characterized by normal plasma PPi levels, but that would benefit from increased plasma levels PPi, for example in order to prevent urinary or salivary stone formation, or to treat urinary or salivary stone formation. In other words, the oral disodium pyrophosphate provided by the current invention may suitable be used in the prevention or treatment of undesired calcification processes in the human body.

In an aspect, the present invention provides the use of disodium pyrophosphate, wherein the disodium pyrophosphate is to be administered in oral form, for use as a medicament, in particular for preventing and/or treating diseases or disorders characterized by calcification, particularly tissue calcification, particularly soft tissue calcification, and/or diseases or disorders characterized by low plasma PPi levels. Thus, the present invention provides the use of disodium pyrophosphate for treating diseases or disorders characterized by calcification, particularly tissue calcification, particularly soft tissue calcification, wherein said disodium pyrophosphate is administered in oral form. The invention also provides a method for reducing calcification, in particular tissue calcification, particularly soft tissue calcification, comprising the step of administering to a subject in need thereof disodium pyrophosphate, wherein said disodium pyrophosphate is administered in oral form.

The subject to be treated may be a human patient exhibiting low levels of pyrophosphate, suffering from a disease or disorder associated with low levels of pyrophosphate, or suffering from a progressive disorder characterized by the accumulation of deposits of calcium and other minerals (mineralization) in, for example elastic fibers (calcification). Calcification happens when calcium builds up in body tissue, blood vessels, or organs. Urinary or salivary stone formation is also to be understood as a form of calcification within the context of the current disclosure. This buildup can harden and disrupt your body's normal processes. Mineralization may occur at the heart, arteries, blood vessels, kidney, spine ligaments, skin, eyes, or the digestive tract. The subject may be of any age and gender, and may have low plasma PPi (although the subject may also have normal plasma PPi levels but would benefit from increased plasma PPi levels). Low plasma PPi may be caused by, for example, congenital deficiencies as taught herein above or others known to result in low plasma PPi levels. Low plasma PPi is also frequently seen in subjects with chronic kidney disease, end-stage renal disease/failure, diabetes mellitus and other conditions. Accordingly, the subject in need of therapy may have ENPP1 deficiency, chronic kidney disease (CKD), end-stage renal disease (ESRD), generalized arterial calcification of infancy (GACI), Pseudoxanthoma elasticum (PXE), Arterial Calcification Due to Deficiency of CD73 (ACDC), Ehlers-Danlos syndrome, arteriosclerosis obliterans, venous calcifications, crystal deposition disorders, calcification resulting from neurological disorders, calcinosis universalis, calcinosis circumscripta, scleroderma, dermatomyositis, systemic lupus erythematosus, hyperparathyroidism, neoplasms, milk-alkali syndrome, hypervitaminosis D, tumoral calcinosis, hypophosphatemic rickets, ossification of the posterior longitudinal ligament of the spine, myocardial ischemia, joint calcification, heterotropic ossification of traumatized muscle, angioid streaks, diabetes mellitus type II, cardiovascular disorder, calciphylaxis, calciphylaxis secondary to chronic kidney disease, calcific uremic arteriolopathy or atherosclerosis. In a preferred embodiment the diseases or disorder characterized by tissue calcification is selected from the group consisting of PXE, GACI, calciphylaxis and calciphylaxis secondary to chronic kidney disease.

Other conditions that can be treated or prevented include urinary or salivary stone (formation) or any other type of undesired calcification in such subject.

The subject is preferably a human, but may also be any other suitable mammal or non-mammal.

Diseases or disorders characterized by tissue calcification, particularly soft tissue calcification, include, but are not limited to, generalized arterial calcification of infancy (GACI), pseudoxanthoma elasticum (PXE), Arterial Calcification Due to Deficiency of CD73 (ACDC), vascular calcification in chronic kidney disease (VCCKD), insulin resistance, hypophosphatemic rickets, ossification of the posterior longitudinal ligament of the spine, myocardial ischemia, joint calcification, heterotropic ossification of traumatized muscle, and angioid streaks. Also treatment of conditions that can be improved by reducing and/or eliminating one or more calcification structures and/or preventing calcification structures from forming in a subject, are within the scope of the present invention. Such conditions include, without limitation, Ehlers-Danlos syndrome, arteriosclerosis obliterans, venous calcifications, crystal deposition disorders, calcification resulting from neurological disorders, calcinosis universalis, calcinosis circumscripta, scleroderma, dermatomyositis, systemic lupus erythematosus, hyperparathyroidism, neoplasms, milk-alkali syndrome, hypervitaminosis D, and tumoral calcinosis.

Generally, the dose of disodium pyrophosphate administered to a subject in need thereof will vary depending upon age, health and weight of the subject, frequency of treatment, and the like. For example, a dose of disodium pyrophosphate may be between about 0.1 mg per kg of body weight and about 1 g per kg of body weight, e.g., between about 0.5 mg per kg of body weight and about 500 mg per kg of body weight, or between about 1 mg per kg of body weight and about 300 mg per kg of body weight, or between about 10 mg per kg of body weight and about 200 mg per kg of body weight, or between about 20 mg per kg of body weight and about 150 mg per kg of body weight. Precise dose and frequency of administration can be determined by a physician skilled in the art.

In a preferred embodiment the disodium pyrophosphate is provided daily. In a preferred embodiment the dosage per day is between 1-500 mg disodium pyrophosphate per kg of body weight, for example between 10-300 mg per kg of body weight, 20-200 mg per kg of body weight or between 30-100 mg per kg of body weight, for example about 50 mg per kg of body weight.

The skilled physician will readily appreciate that certain factors may influence the dose or dosage required to effectively treat a subject, including, but not limited to, the severity of the disease, previous treatments, the general health and/or age of the subject, and other diseases present in the subject.

The amount of disodium pyrophosphate may be in the form of a unit-dosage comprising all of the therapeutically effective amount, or may be contained in multiple dosage forms. The disodium pyrophosphate may be administered once daily, twice daily, or 3, 4, 6, 7, 8, 9, or 10 times daily, or the like. It will be appreciated that the effective dosage of disodium pyrophosphate used for the treatment taught herein may increase or decrease over the course of the treatment.

The oral disodium pyrophosphate may be administered to a subject for a period of time determined by a skilled physician. In one embodiment, e.g., for certain hereditary calcification disorders, the period of time will be the remainder of the subject's life span.

In one embodiment, the subject is an infant. The subject may be between 1 month and 24 months in age, less than 1 year of age, less than 2 years of age, less than 3 years of age, less than 4 years of age, less than 5 years of age, or less than 6 years of age.

In an embodiment, the level of blood/plasma PPi in a subject prior to treatment is less than about 80%, such as less than about 70%, less than about 60%, less than about 50%, less than about 40%, less than about 30%, less than about 25%, less than about 20%, less than about 15%, less than about 10%, less than about 5%, less than about 3%, less than about 2% or less than about 1% of normal levels of PPi observed in a healthy human subject. In an embodiment, a subject shows no measurable level of blood/plasma PPi prior to treatment.

In an embodiment, the disodium pyrophosphate may be administered in conjunction with a pharmaceutically acceptable carrier, diluent, or excipient. As according to the invention the disodium pyrophosphate is administered orally, it may be presented in any form suitable for such administration, e.g. in the form of tablets, capsules, powders, syrups or solutions. In one preferred embodiment, the disodium pyrophosphate is administered in the form of a solid pharmaceutical entity, suitably as a tablet or a capsule. In one preferred embodiment, the disodium pyrophosphate is administered in the form wherein the disodium pyrophosphate is not in a solution. Methods for the preparation of solid pharmaceutical compositions or preparations are well known in the art. Thus, tablets may be prepared by mixing the active ingredient with conventional adjuvants, fillers and diluents and subsequently compressing the mixture in a suitable tableting machine. Examples of adjuvants, fillers and diluents comprise cornstarch, lactose, talcum, magnesium stearate, gelatin, gums, and the like. Typical fillers are selected from lactose, mannitol, sorbitol, cellulose and microcrystalline cellulose. Any other adjuvant or additive such as colorings, aroma, preservatives, etc, may also be used provided that they are compatible with the active ingredient disodium pyrophosphate. In an embodiment, the oral disodium pyrophosphate is in the form of an extended release, slow release or delayed release formulation.

In an embodiment, the disodium pyrophosphate may be included in a food or food supplement product, e.g., a sweet or chewing gum, or the like.

In an embodiment, the disodium pyrophosphate is in the form of an oral pharmaceutical composition.

The oral disodium pyrophosphate may be administered alone or in combination with other agents. The disodium pyrophosphate may be administered before, after, or concurrently with such other agents or can be co-administered with other known therapies.

In an embodiment, the present invention pertains to a method for increasing plasma inorganic pyrophosphate levels in a subject in need thereof, comprising the step of administering to the subject a therapeutically effective amount of disodium pyrophosphate, wherein said disodium pyrophosphate is administered in oral form.

In an embodiment, the present disclosure pertains to a method for preventing and/or reducing calcification, particularly tissue calcification, particularly soft tissue calcification, and/or diseases or disorders characterized by low plasma PPi levels, in a subject in need thereof. The method is based on the surprising finding that oral disodium pyrophosphate can be administered to a subject, for example a person that has low plasma PPi levels, to cause a transient increase in plasma PPi in the subject, which can inhibit calcification, particularly tissue calcification, particularly soft tissue calcification, in the subject. Since the increase in plasma PPi is transient, therapy can be tailored to inhibit undesirable or pathological tissue calcification, without inhibiting bone calcification or inducing osteomalacia.

In an embodiment, the disclosure relates to a method for reducing calcification, particularly tissue calcification (e.g. soft tissue calcification) in a subject in need thereof, by administering to the subject one or more doses of disodium pyrophosphate. Each dose may contain an amount of disodium pyrophosphate that is sufficient to achieve a transient increase in plasma PPi in the subject.

In an embodiment, the PPi level in the blood/plasma returns to its base level within about 24 hours, such as within 18 hours, within 12 hours, within 6 hours, or within 4 hours, after administration of the dose disodium pyrophosphate. The time period between the administration of each dose may vary. For example, oral disodium pyrophosphate may be administered twice a day (or one to ten times a day) a day, daily, once every two days, once every three days, once every four days, or the like.

In an embodiment, each dose of oral disodium pyrophosphate that is administered to the subject contains an amount of disodium pyrophosphate sufficient to achieve a transient increase in plasma PPi level, which may have a peak that is above 200%, or that is between about 40% and about 200%, such as between about 50% and 150%, between about 60% and about 125%, between about 70% and about 100%, between about 80% and about 90%, of the plasma PPi level observed in healthy subjects.

In an embodiment, the transient increase in plasma PPi level after oral administration of disodium pyrophosphate is maintained for at least about 10 minutes, 15 minutes, 30 minutes, 1 hour, or the like. Further, it is preferred that the plasma PPi level returns to its base level within about 24 hours, such as within about 18 hours, 12 hours, 6 hours, 5 hours, 4 hours, 3 hours, 2 hours, 1 hour or less after administration of the dose disodium pyrophosphate.

In case the subject is characterized by low plasma PPi, the low plasma PPi levels in a subject prior to treatment may be about 50% or less, 40% or less, 30% or less, 20% or less, or 10% or less, of the plasma PPi levels observed in a healthy subject.

Calcification, particularly tissue calcification is a progressive process, and individuals born with congenital deficiencies leading to low plasma PPi levels may not show tissue calcification for several years. In order to reduce or minimize calcification in such subjects, therapy should be initiated as early as possible, preferably even before tissue calcification is noticed.

In subjects with low plasma PPi levels which do not have congenital deficiencies, therapy should begin as soon as practicable; i.e. soon after the diagnosis of the conditions, such as CKD or ESRD.

It will be understood that all details, embodiments and preferences discussed with respect to one aspect of embodiment of the invention is likewise applicable to any other aspect or embodiment of the invention and that there is therefore not need to detail all such details, embodiments and preferences for all aspect separately.

Having now generally described the invention, the same will be more readily understood through reference to the following examples which is provided by way of illustration and is not intended to be limiting of the present invention. Further aspects and embodiments will be apparent to those skilled in the art.

### EXAMPLES

### Example 1

### General Introduction

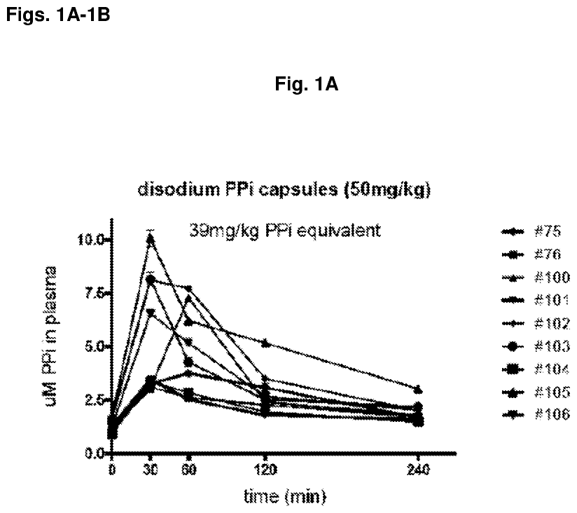

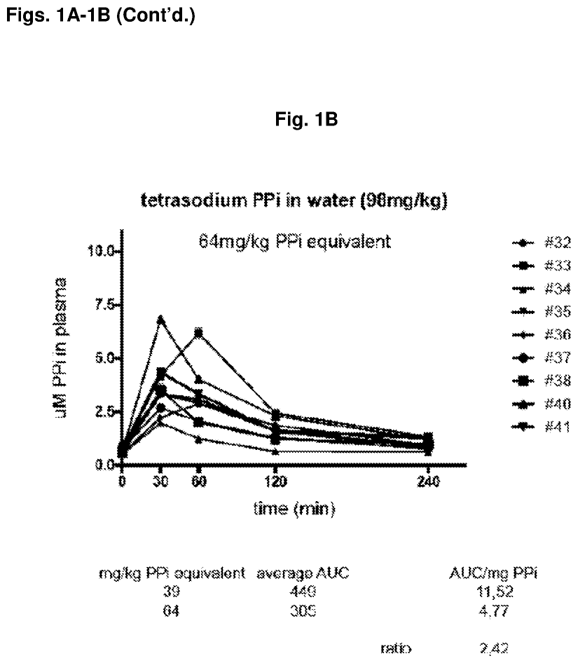

In this experiment PPI bioavailability was compared between tetrasodium PPi and disodium PPi in different human subjects (as indicated by # in FIG. 1). As may be witnessed from FIG. 1, PPi is much better bioavailable in plasma after oral disodium PPi than after oral tetrasodium PPi uptake of disodium PPi.

The area under the curve (AUC) values as well as the calculated AUC/mg PPi value are both higher for disodium PPi than for tetrasodium PPi. The ratio between the calculated AUC/mg PPi for disodium PPi and for tetrasodium PPi is 2.4.

### Example 2

A 59-year-old male having medication for hypertension, type 2 diabetes and hypercholesterolemia, was referred to vascular surgeon due to rest pain in the right lower limb. He has a 40-year history of smoking. Previous suspicion of Pseudoxanthoma elasticum (PXE) on basis of central vision loss and typical skin lesion in the neck was confirmed by skin biopsy and later by homozygous mutation of ABCC6 gene (c.3421C>T, p.Arg1141). The patient has suffered from intermittent claudication, which has then progressed to rest pain in the right limb over the last two months.

The ankle-brachial pressure index (ABI) at rest was 0.26 on the right and 0.48 on left side. The Walking Impairment Questionnaire (WIQ) score was 0.19. Magnetic resonance angiography (MRA) showed bilateral occlusion of the common and the superficial femoral artery, as well as occlusion of the left external iliac artery.

Patient had critical ischaemia in his right limb requiring revascularization and he was treated with the right common femoral artery endarterectomy and profundoplasty using the anterior accessory saphenous vein as a patch. The patient quit smoking before the procedure.

As PXE patients have an increased risk of spontaneous gastrointestinal bleeding, aspirin medication was not used preoperatively. To prevent re-occlusion, an experimental pyrophosphate treatment was initiated two weeks before the operations with a fasting per oral once daily dose of 3600 mg (44 mg/kg) disodium pyrophosphate powder dissolved into 2 dl of water. With the present therapy the patients absorbs extra 0.8 mg sodium and is informed to decrease daily salt intake.

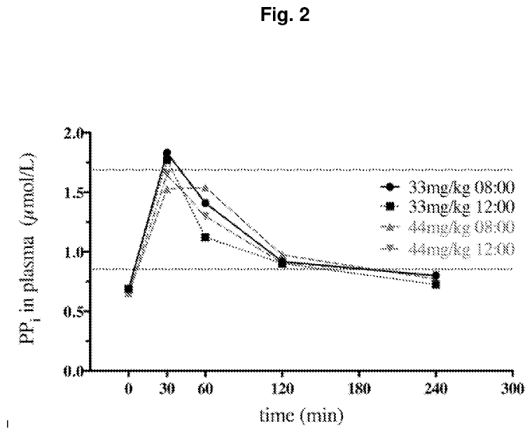

Before instituting treatment, absorption of disodium pyrophosphate with two different doses was tested at ward by a two-day protocol. Fasting plasma pyrophosphate concentration was below normal, 0.7 μmol/l (healthy controls 0.8-1.6 μmol/l). A single dose of pyrophosphate increased the concentration of plasma pyrophosphate to the normal fasting range of healthy controls for 180 minutes (FIG. 2).

The surgical procedure and recovery were uncomplicated. At one-month control visit, the operated right limb was asymptomatic. The patient experienced only mild claudication of the other limb. The ABI was 0.70 on the right side and 0.52 on the left side. Low-dose aspirin treatment (50 mg per day) was started as the patient had no history of gastrointestinal bleeding.

Three months after the operation, the right limb was still asymptomatic, with an ABI of 0.82. MRA showed no re-stenosis in the operated area. The collateral network had increased on both sides as analysed by an independent radiologist. Safety laboratory tests were normal and the patient experienced no significant side effects. Aspirin and PPi therapy were continued unaltered.

At six-month and one-year control exams he was measured elevated systemic blood pressure and medication for it was increased. There were no side effects of the disodium pyrophosphate treatment and the supplementation therapy has been continued. Recent systemic blood pressure have stayed in recommended limits. One year postoperatively the improved clinical situation remains without signs of re-stenosis on computed tomography angiography (CTA). The WIQ score has improved to 0.89.

This is the first report to show that low fasting PPi level can be normalized for three hours by oral disodium pyrophosphate administration. Individualized per oral disodium pyrophosphate treatment was started with a dose (44 mg/kg).

During the one-year follow-up, the operated limb has been asymptomatic and the operated area has not re-occluded. Furthermore, the ABI and WIQ have improved from the baseline level, and the collateral network has increased in MRA. The new disodium pyrophosphate supplementation therapy shows promise, and the side effects seem negligible.

Having now fully described this invention, it will be appreciated by those skilled in the art that the same can be performed within a wide range of equivalent parameters, concentrations, and conditions without departing from the spirit and scope of the invention and without undue experimentation.

All references cited herein, including journal articles or abstracts, published or corresponding patent applications, patents, or any other references, are entirely incorporated by reference herein, including all data, tables, figures, and text presented in the cited references. Additionally, the entire contents of the references cited within the references cited herein are also entirely incorporated by references.

Reference to known method steps, conventional methods steps, known methods or conventional methods is not in any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein.

It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art./pct.

1. Disodium pyrophosphate for use as a medicament, wherein said disodium pyrophosphate is administered in oral form.

2. Disodium pyrophosphate for use in preventing and/or treating diseases or disorders characterized by calcification, particularly tissue calcification, particularly soft tissue calcification, or diseases or disorders characterized by low plasma inorganic pyrophosphate (PPi) levels, wherein said disodium pyrophosphate is administered in oral form.

3. Disodium pyrophosphate for use according to claim 2, wherein the soft tissue calcification is vascular calcification such as arterial calcification or intimal calcification.

4. Disodium pyrophosphate for use according to claim 2, wherein the tissue calcification is in a subject having ENPP1 deficiency, chronic kidney disease (CKD), end-stage renal disease (ESRD), generalized arterial calcification of infancy (GACI), Pseudoxanthoma elasticum (PXE), Arterial Calcification Due to Deficiency of CD73 (ACDC), Ehlers-Danlos syndrome, arteriosclerosis obliterans, venous calcifications, crystal deposition disorders, calcification resulting from neurological disorders, calcinosis universalis, calcinosis circumscripta, scleroderma, dermatomyositis, systemic lupus erythematosus, hyperparathyroidism, neoplasms, milk-alkali syndrome, hypervitaminosis D, tumoral calcinosis, hypophosphatemic rickets, ossification of the posterior longitudinal ligament of the spine, myocardial ischemia, joint calcification, heterotropic ossification of traumatized muscle, angioid streaks, diabetes mellitus type II, cardiovascular disorder, calciphylaxis, calciphylaxis secondary to chronic kidney disease, calcific uremic arteriolopathy or atherosclerosis.

5. Disodium pyrophosphate for use according to claim 2, wherein the disodium pyrophosphate is to be administered to a human subject.

6. Disodium pyrophosphate for use according to claim 2, wherein the disodium pyrophosphate is to be administered daily.

7. Disodium pyrophosphate for use according to claim 6, wherein the daily dose disodium pyrophosphate administered is 10-500 mg disodium pyrophosphate per kilogram bodyweight.

8. A method for preventing and/or reducing calcification, particularly tissue calcification, particularly soft tissue calcification, and/or diseases or disorders characterized by low plasma PPi levels, comprising the step of administering to a subject in need thereof a therapeutically effective amount of disodium pyrophosphate, wherein said disodium pyrophosphate is administered in oral form.

9. The method according to claim 8, wherein the soft tissue calcification is vascular calcification such as arterial calcification or intimal calcification.

10. The method according to claim 8, wherein the disodium pyrophosphate is sufficient to achieve a transient increase in plasma PPi level in the subject.

11. The method according to claim 8, wherein the transient increase in plasma PPi level is characterized by a PPi level that is at least about 40% of the plasma PPi level in a healthy subject.

12. The method according to claim 10, wherein the transient increase in plasma PPi level is maintained for at least about 15 minutes.

13. The method according to claim 8, wherein the subject has a disease or disorder characterized by low plasma PPi levels, e.g., chronic kidney disease (CKD), end-stage renal disease (ESRD), generalized arterial calcification of infancy (GACI), hypophosphatemic rickets, heterotropic ossification of traumatized muscle, a cardiovascular disorder, calciphylaxis, calciphylaxis secondary to chronic kidney disease, calcific uremic arteriolopathy, atherosclerosis and/or pseudoxanthoma elasticum (PXE), Arterial Calcification Due to Deficiency of CD73 (ACDC), Ehlers-Danlos syndrome, arteriosclerosis obliterans, venous calcifications, crystal deposition disorders, calcification resulting from neurological disorders, calcinosis universalis, calcinosis circumscripta, scleroderma, dermatomyositis, systemic lupus erythematosus, hyperparathyroidism, neoplasms, milk-alkali syndrome, hypervitaminosis D, tumoral calcinosis, or diabetes mellitus type II.

14. The method according to claim 13, wherein the subject has GACI or PXE.

15. The method according to claim 8, wherein the daily dose disodium pyrophosphate administered is 10-500 mg disodium pyrophosphate per kilogram bodyweight.

16. A method for increasing plasma inorganic pyrophosphate levels in a subject in need thereof, comprising the step of administering to the subject a therapeutically effective amount of disodium pyrophosphate, wherein said disodium pyrophosphate is administered in oral form.
